(19) United States
(12) Patent Application Publication
(10) Pub. No.: US 2006/0280664 A1
(43) Pub. Date: Dec. 14, 2006

(54) Electrolytic sterilizing atomization device

(76) Inventors: Chuan-Pan Huang, Tainan City (TW); Chen-Lung Huang, Tainan City (TW)

Correspondence Address: J.C. Patents;Suite 250, 4 Venture, Irvine, CA, 92618, US

(21) Appl. No.: 11/131,658

(22) Filed: May 17, 2005

(51) Int. Cl. A61L 2/00

(52) U.S. Cl. 422/292; 422/22; 204/194; 210/243; 422/20; 210/206

(57) ABSTRACT

An electrolytic sterilizing atomization device comprises a main body and an electrolytic trough. A liquid tank, an atomization room and an atomization are provided on the main body. The electrolytic trough is in fluid communication with the liquid tank. An electrode plate is provided in the electrolytic trough for atomizing a chlorine-containing liquid supplied from the liquid tank, and generating a hypochlorous acid-containing. The atomization component can atomize the liquid containing hypochlorous acid. The atomized liquid is sprayed to environment through an atomization room.

### FIELD OF THE INVENTION

The present invention provides an electrolytic sterilizing atomization device, particularly, one to store a liquid with chlorine (such as running water) in the liquid tank of a main body, and to electrolyze the liquid with the electrode plate designed in the electrolytic trough at the side of the liquid tank to generate a hypochlorous acid(HClO)-containing liquid, so that we can atomize the liquid with hypochlorous acid with an atomization component and transmit the atomized liquid to the working environment, and the electrolytic sterilizing atomization device can be achieved, which has sterilization function against the working environment.

### BACKGROUND OF THE INVENTION

Generally, the prior atomization devices applied to working environment use a negative pressure siphon air pump, such as Taiwan Utility Model Patent No. M247213, entitled Vibration Essence Oil Diffuser. This kind of diffuser mainly is composed of an air pump whose air conduit outlet connects to the pipe column of an oil cup; and because an air inlet pipe and suction tube were designed, it only can atomize the liquid in the oil bottle during air pump sends air into the liquid in the oil cup. Therefore, this prior patent only can atomize the liquid with fixed components and transmit them. However the present invention takes the facile running water and electrolyzes it firstly for generating the liquid with hypochlorous acid, and its sterilization function and utility can be achieved.

### SUMMARY OF THE INVENTION

The purpose of the present invention is to provide an electrolytic sterilizing atomization device. In the electrolytic trough of the electrolytic sterilizing atomization device, an electrode plate is provided. A chlorine-containing liquid is electrolyzed by means of the electrode plate to generate a hypochlorous acid-containing liquid. The liquid with hypochlorous acid is then atomized and transmitted into the environment.

The present invention has the following features:

1. The present invention provides an electrolytic sterilizing atomization device, in which an atomization component is provided at the lower part of an atomization room of the main body, and the atomization component can be a negative pressure siphon air pump or a ultrasonic oscillator.

2. The present invention provides an electrolytic sterilizing atomization device, in which an electrode plate is provided in an electrolytic trough for electrolyzing a chlorine-containing liquid in the liquid tank, and generating a hypochlorous acid-containing liquid.

### BRIEF DESCRIPTION OF DRAWINGS

FIG. 1 is a sectional view of embodiment 1 of the present invention.

FIG. 2 is a sectional view of embodiment 2 of the present invention.

FIG. 3 is a section view of embodiment 3 of the present invention.

FIG. 4 is a sectional view of embodiment 4 of the present invention.

### DETAILED DESCRIPTION OF PREFERRED EMBODIMENTS

Therefore, for better understanding of the present invention, descriptions of the invention in connection with the drawings are given as follows:

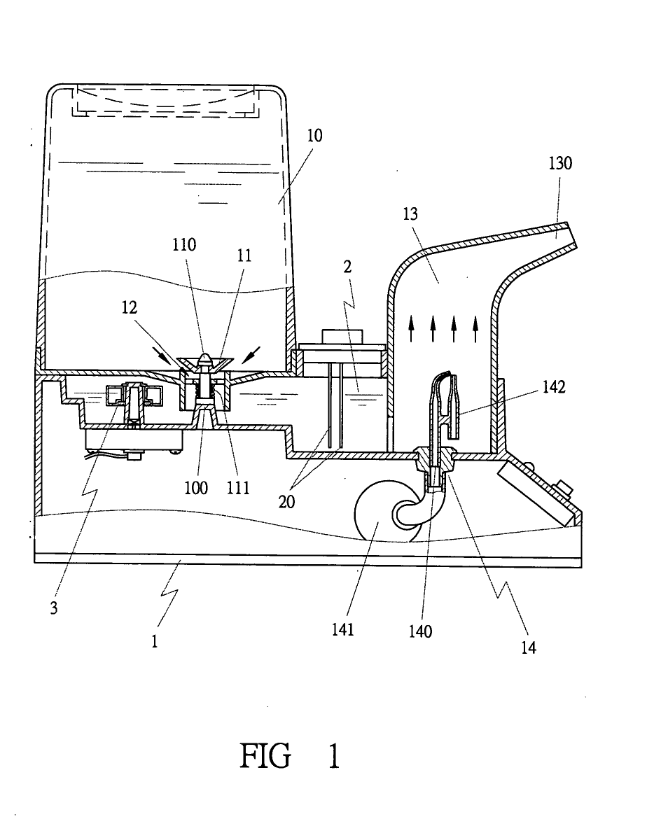

As shown in FIG. 1, it is the embodiment 1 of the electrolytic sterilizing atomization device of the present invention, which comprises a main body 1 and an electrolytic trough 2.

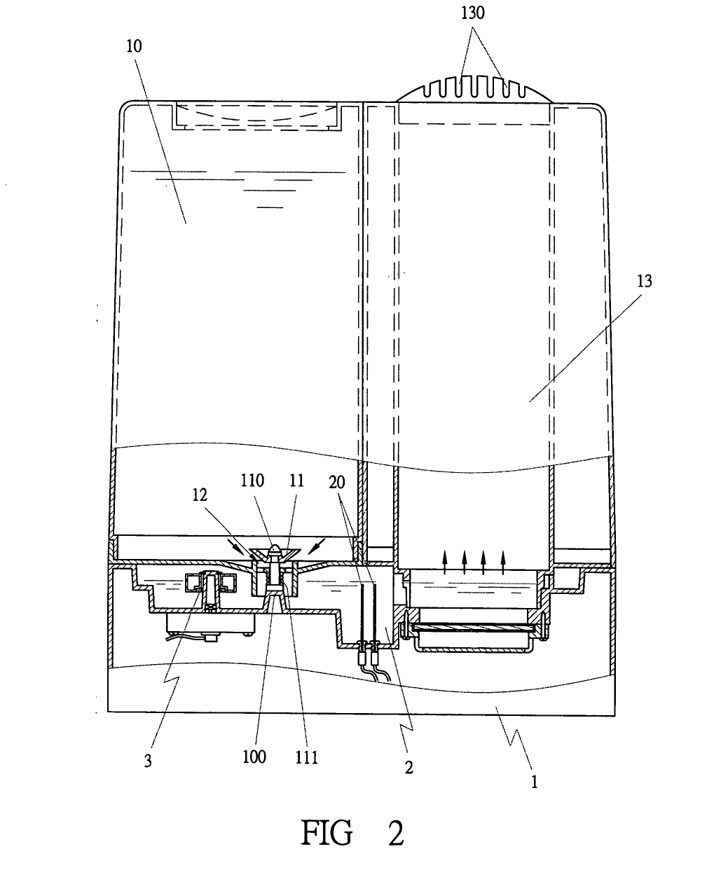

A liquid tank 10 is provided on the upper side of the main body 1, and the liquid tank 10 is detachable from the main body 1 for convenient filling of a chlorine-containing liquid (such as running water), a relative large quantity of liquid can be stored in liquid tank 10. An outlet valve 11 is designed at the lower part of the liquid tank 10, the outlet valve 11 has a valve handle 110. The valve handle 110 extends through an outlet 12 and a spring 111. The lower end of the valve handle 110 reaches an outlet valve column 100 so as to control the liquid level of an electrolytic trough 2 and maintain a proper liquid level of an atomization room 13. An inspection component 3 is provided under the liquid tank 10, which can be a photoelectric or magnetic sensor, such as Reed sensor, Hall IC and so on (because the inspection component is well known, no details are given in the drawings). Furthermore, an atomization room 13 is provided in the main body 1, the atomization room 13 has a spray outlet. An atomization component 14 is provided at the lower part of the atomization room 13, and the atomization component 14 can be a negative pressure siphon air pump (as shown in FIG. 1) or a ultrasonic oscillator (as shown in FIG. 2), or other suitable known atomizing unit. The negative pressure siphon air pump must be designed with an air inlet pipe 140 connecting to a pump 141, and a suction tube 142 is designed at the side of the air inlet pipe 140, so that the liquid sucked by the siphon from the suction tube 142 can be atomized while the air inlet pipe 140 takes air in and, after atomization, transmitted into the working environment with the power the abovementioned pump 141 transmits into the atomization room 13.

Electrolytic trough 2 has an electrode plate 20 that can be installed on the top of the electrolytic trough 2 and extends downward (as shown in FIG. 1), or on the bottom of the electrolytic trough 2 and extends upward (as shown in FIG. 2), or on other suitable locations and orientations in the electrolytic trough 2. Thus, the electrode plate 20 in the electrolytic trough 2 can electrolyze the chlorine-containing liquid supplied from the liquid tank 10, and generate a hypochlorous acid-containing liquid, which would be atomized by the atomization component 14 and transmitted to the working environment for sterilization.

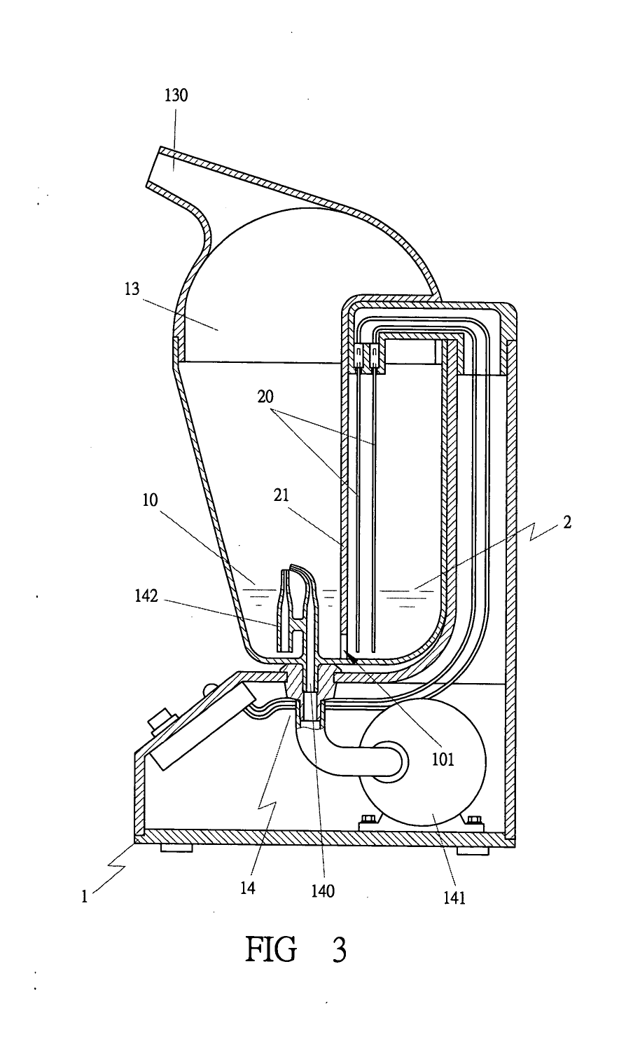

FIG. 3 shows an electrolytic sterilizing atomization device according to the embodiment 3 of the present invention, which mainly is different from the embodiment 1 and 2 in appearance. and comprises a main body 1 and a electrolytic trough 2. A liquid tank 10 is provided on the upper side of the main body 1, and the liquid tank 10 is detachable from the main body 1 for convenient filling of a chlorine-containing liquid (such as running water). A relative large quantity of liquid can be stored I liquid tank 10. At the upper portion of the liquid tank 10, a space is preserved as an atomization room 13, in which a spray outlet 130 is provided while an atomization component 14 is provided under the atomization room 13. The atomization component can be a negative pressure siphon air pump (as shown in FIG. 1) or a ultrasonic oscillator (as shown in FIG. 2), or other suitable known atomizing unit. The negative pressure siphon air pump comprises an air inlet pipe 140 connecting to a pump 141, and a suction tube 142 is designed at the side of the air inlet pipe 140, so that the liquid sucked by the siphon from the suction tube 142 can be atomized while the air inlet pipe 140 takes air in, and, after atomization, transmitted into the working environment with the power the abovementioned pump 141 transmits into the atomization room 13.

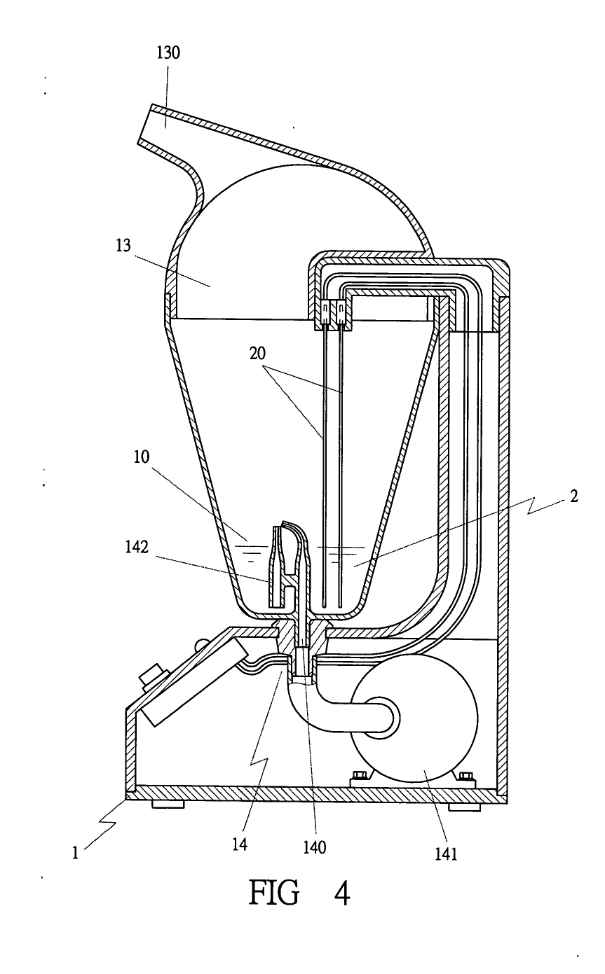

A baffle wall 21 is designed between the electrolytic trough 2 and the liquid tank 10 (as shown in FIG. 3). A passage 101 is available between the electrolytic trough 2 and the liquid tank 10. The baffle wall 21 can be omitted (as shown in FIG. 4). An electrode plate 20 is designed in the electrolytic trough 2 or in the combined body of the electrolytic trough 2 and the liquid tank 10 as shown in FIG. 4, which can be installed in any suitable location in any suitable orientation in the electrolytic trough 2. Thus, the electrode plate 20 in the electrolytic trough 2 can electrolyze a chlorine-containing liquid in the liquid tank 10, and generate a hypochlorous acid-containing liquid, which would be atomized by the atomization component 14 and transmitted to the working environment for sterilization.

What the claimed is:

1. An electrolytic sterilizing atomization device, comprises:
a main body;
a liquid tank coupled to the man body;
an atomization room coupled to the main body;
an atomization component coupled to the main body;
an electrolytic trough comprising an electrode plate for electrolyzing a chlorine-containing liquid supplied by the liquid tank, and generating a hypochlorous acid-containing liquid.

2. The electrolytic sterilizing atomization device, as recited in claim 1, wherein the atomization component comprises a ultrasonic oscillator.

3. The electrolytic sterilizing atomization device, as recited in claim 1, wherein the atomization component comprises a negative pressure siphon air pump.

4. The electrolytic sterilizing atomization device, as recited in claim 1, wherein the electrolytic trough is located below the liquid tank.

5. The electrolytic sterilizing atomization device, as recited in claim 1, wherein the atomization component is positioned at a lower portion of the atomization room.

6. An electrolytic sterilizing atomization device, comprising:
a main body;
a liquid tank coupled to the main body;
an atomization room coupled to the main body;
an atomization component coupled to the main body;
an electrolytic trough in fluid communication with the liquid tank, wherein a baffle wall is provided between the electrolytic trough and the liquid tank, and an electrode plate positioned in the electrolytic trough for electrolyzing a chlorine-containing liquid supplied from the liquid tank, and generating a hypochlorous acid-containing liquid.

7. The electrolytic sterilizing atomization device, as recited in claim 6, wherein the atomization component comprises a ultrasonic oscillator.

8. The electrolytic sterilizing atomization device, as recited in claim 6, wherein the atomization component comprises a negative pressure siphon air pump.

9. The electrolytic sterilizing atomization device, as recited in claim 6, wherein the electrolytic trough is located beside the liquid tank.

10. The electrolytic sterilizing atomization device, as recited in claim 6, wherein the atomization component is positioned at a lower portion of the liquid tank.

11. An electrolytic sterilizing atomization device, comprising:
a main body;
a liquid tank coupled to the main body;
an atomization room coupled to the main body;
an atomization component coupled to the main body;
an electrode plate positioned in the liquid tank for electrolyzing a chlorine-containing liquid in the liquid tank, and generating a hypochlorous acid-containing liquid.

12. The electrolytic sterilizing atomization device, as recited in claim 11, wherein the atomization component is positioned at a lower portion of the liquid tank.
